(19) United States
(12) Patent Application Publication
(10) Pub. No.: US 2006/0134615 A1
(43) Pub. Date: Jun. 22, 2006

(54) Method for the simultaneous detection of HPV RNA and DNA in a sample

(76) Inventors: James Linder, Acton, MA (US); Brian Lentrichia, Acton, MA (US); J. Lawrence Burg, Framingham, MA (US)

Correspondence Address: Theodore Allen, Esq.;Cytye Corporation, 250 Campus Drive, Marlborough, MA, 01752, US

(21) Appl. No.: 11/297,689

(22) Filed: Dec. 8, 2005

Related U.S. Application Data
(60) Provisional application No. 60/637,558, filed on Dec. 20, 2004.

(51) Int. Cl. C12Q 1/70; C12Q 1/68

(52) U.S. Cl. 435/5; 435/6

(57) ABSTRACT

A method is provided for the simultaneous detection of human papillomavirus DNA and RNA in a sample.

### FIELD OF THE INVENTION

A method is provided for the simultaneous detection of human papillomavirus DNA and RNA in a sample.

### BACKGROUND OF THE INVENTION

Epidemiological studies have long implicated the human papillomavirus (HPV) as a major cause of cervical neoplasia and cancer. Epidemiologic data are accumulating which show that the human papillomavirus (HPV) is strongly associated, directly or indirectly, with cervical cytologic abnormalities, such as cervical intraepithelial neoplasia (CIN), squamous cell carcinoma, adenocarcinoma, and carcinoma in situ (CIS) (Wolf JK, 2003). In general, these dysplasia are found in the transition zone of the cervix and are graded from mild to severe (I to III), based on the extent to which neoplastic cells extend from the basal layer to the epithelial surface. Complete replacement of the epithelium by neoplastic cells is termed carcinoma in situ (CIS).

Although current data strongly supports the close association between HPV and cervical neoplasia and shows that many individuals clearly have HPV DNA (i.e., are infected with HPV), transiently and/or latently, it also makes it clear that women in whom HPV is present in cervical cells do not always progress to more serious dysplasia, even in persistent infections (Wright et al. 2004). Thus, simple detection of HPV DNA in cervical specimens lacks discriminatory predictive value for identification of women at risk for disease.

A variety of methods for detecting HPV have been devised. Many of those rely on the detection of unique sequences in the HPV genome. For example, DNA or RNA probes complementary to a portion of the genes of a particular high risk HPV strain have been reported in the art, e.g., in U.S. Pat. No. 4,849,332 to Lorincz, incorporated herein by reference, as useful in screening for the presence of a particular strain of high-risk HPV in patient samples. U.S. Pat. No. 5,705,627 to Manos et al., incorporated herein by reference, reports use of polymerase chain reaction (PCR) to amplify and detect HPV DNA using degenerate or mixed consensus primers, followed by typing using a mixture of genotype-specific DNA probes. Other examples of using consensus primers can be found in U.S. Pat. No. 5,364,758 to Meijer et al., and Kleter, B. et al. 1998). At least 60 types of HPV, isolated from various parts of the human body, have been documented, and more than 20 of these types have been shown to be associated with the genital mucosa. (E. M. de Villiers, 1989). These references are also incorporated herein by reference to the extent applicable.

The HPVs that infect the anogenital tract can be classified as either “high risk” or “low risk” based on their association with cancer. HPV types 16 and 18 are the most common of the high risk group, while HPV type 6 and 11 are among the low risk types. Approximately 90% of cervical cancers contain HPV DNA of the high risk types, and these same DNAs are found in the precancerous epithelial lesions (zur Hausen and Schneider, “The Role of Papillomaviruses in Human Anogential Cancer”, in The Papovaviridea, Vol. 2, Plenum, New York; 1987; Riou et al., Lancet, 335:1171-1174 (1990)). The low risk types are associated primarily with benign lesions such as condyloma acuminata and are only rarely found associated with cancers. Transfection of DNA of the high risk HPVs results in the extended life span and immortalization of primary human keratinocytes and fibroblasts in cell culture, whereas DNA of the low risk types does not (Durst et al., Oncogene, 1:251-256 (1987); Pirisi et al., J. Virol., 61:1061-1066 (1987); Schlegel et al., EMBO J., 7:3181-3187 (1988)).

Although there are clear benefits to the use of HPV DNA testing, there are potential problems as well. Because infection of HPV is so common (at least 80 percent of women will at least one HPV infection by the age of 50; see 2004 CDC fact sheet), large numbers of women who are tested for HPV DNA will be identified as HPV positive, but only a small number of these women will actually develop a cervical lesion. Therefore, it is estimated that approximately 10-20% of the adult population would be infected with transient, clinically insignificant HPV (Wright et al. 2004).

### SUMMARY OF THE INVENTION

The present invention describes a method for the simultaneous detection of both HPV RNA and DNA in a sample. Methods for the detection of HPV RNA, such as the polymerase chain reaction using a reverse transcriptase enzyme (RT-PCR), and nucleic acid sequence based amplification (NASBA), are well know. The presence of HPV DNA is not necessarily an indication of an active infection. The presence of RNA transcripts (mRNA) has been suggested to be a much better indication of active and persistent infections, especially those transcripts from the E6 and E7 oncogenes (Ylitalo et al. 2000). However, since HPV can exist in an inactive or latent state (i.e., with little or nor transcription), an assay designed to detect only HPV RNA would potentially miss an HPV viral infection which, at a later date, could become active. There exists the need for a method of specifically detecting the clinically significant HPV infections or to further differentiate the clinically significant HIPV infections from the clinically insignificant infections. Thus, a unique method comprising the simultaneous detection of human Papillomavirus (HPV) RNA and DNA from a specimen infected with the virus in provided.

In one embodiment, methods are used to detect nucleic acids associated with HPV, which in turn provides a basis for medical diagnosis and treatment. Methods of the invention solve the problems in the art through the detection of both HPV RNA and DNA at the same time from the same specimen sample rather than independently from two different samples from the same specimen.

In one embodiment of the invention, methods are provided for detecting the presence of specific HPV target nucleic acids in a biological sample using RNA and DNA probes. Preferred methods comprise suspending sample cells in a solution; isolating total nucleic acid from the sample cells; contacting a target DNA sequence with at least one probe that is substantially complementary to at least a portion of said target nucleic acid, the detection of which is desired; contacting a target RNA sequence with at least one probe that is substantially complementary to at least a portion of a nucleic acid, the detection of which is desired; and detecting hybridization between the probes and the target sequences. Preferably, the solution for suspending the sample cells contains an alcohol in an amount sufficient to fix sample cells without coagulation, an anti-clumping agent, and a buffer that maintains the solution at a pH within a range of about 4 to 7.

In another embodiment of the invention, the probes are labeled with a detectable marker, such as a fluorophore (e.g., molecular beacons).

In yet another embodiment, the presence of the target DNA sequence in the sample cells is diagnostic of an HPV infection, the presence of the target RNA sequence in the sample cells is diagnostic of an active HPV infection and may also be indicative of risks of cancer, such as risks associated with adenocarcinoma and squamous cell carcinoma of the cervix.

In still another embodiment, the presence of the target DNA sequence is indicative of the presence of a particular type of HPV. In more preferred embodiments, the target nucleic acid sequence is indicative of the presence of high risk HPV strains selected from types 16, 18, 31, 33, 35, 39, 45, 51, 52, 56, 58, 59, 68 and 70.

In another embodiment, methods of the invention further comprise capturing the target nucleic acid onto a solid support using DNA-DNA, DNA-RNA, or RNA-DNA interactions.

Alternative embodiments of the invention include methods for detecting the presence of a target nucleic acids of HPV in a sample, comprising: capturing target DNA sequences onto a solid support with at least one probe that is substantially complementary to at least a portion of target DNA sequences of one or more HPV types, capturing target RNA sequences onto a solid support with at least one probe that is substantially complementary to at least a portion of target RNA sequences of one or more HPV types, and then detecting hybridization between the probes and the target nucleic acids. Other embodiments relate to methods for in situ detection of target DNA and RNA sequences of HPV in a sample by transferring suspended sample cells uniformly onto a surface; hybridizing target sequences of one or more HPV types contained in the cells with at least one probe that is substantially complementary to portions of a target DNA sequence and a target RNA sequence of one or more HPV types; and detecting hybridization between the probes and a target sequences.

Another embodiment of the invention provides methods of detecting target nucleic acids comprising amplifying target sequences. Such methods generally comprise providing a sample that may comprise DNA and RNA target sequences from at least one selected strain of an organism. Also provided are multiple primers substantially complementary to portions of target sequences from the selected strains of the organism. The sample is exposed to at least one analog probe that is sufficiently complementary to a portion of a DNA target sequence and to at least one analog probe that is sufficiently complementary to a portion of an RNA target sequence. The nucleic acid from at least one selected strain is amplified between the multiple primers and detection of such amplification product indicates the presence of at least one target nucleic acid in the sample.

In still another embodiment, methods of the invention are used to selectively amplify high-risk strains of an infectious organism. In such methods, consensus primers for amplification of a preselected region of the infectious organism's DNA and RNA are used. Alone, these primers are capable of amplifying target sequences from most or all of the strains or variants of the disease organism. However, in methods of the invention, amplification of non-selected strains or variants is blocked such that any amplicon produced is representative of only the unblocked strains or variants. For example, methods of the invention may be used to selectively detect active infections of high-risk strains of HPV. An amplification reaction is conducted in the presence of HPV consensus primers and one or more nucleic acid blocking probes that hybridize only to a region at or between the primers in non-selected low-risk strains of HPV. The blocking probes prevent amplification of DNA and RNA target sequences from low-risk strains (i.e., full-length amplification between primers is prevented), while DNA and RNA target sequences from high-risk strains to which the blocking probes do not hybridize are amplified.

Other features and advantages of the invention will be apparent from the following detailed description and claims.

Definitions

In the description that follows, a number of terms used in recombinant DNA technology are extensively utilized. In order to provide a clear and consistent understanding of the specification and claims, the following definitions are provided.

As used herein, the term “amplification” refers to any in vitro method for increasing the number of copies of a nucleic acid sequence (either DNA or RNA) with the use of an enzyme. Nucleic acid amplification results in the incorporation of nucleotides into a DNA (or RNA) molecule or primer thereby forming a new DNA (or RNA) molecule complementary to a nucleic acid template. The newly formed DNA (or RNA) molecule can be used as a template to synthesize additional DNA (or RNA) molecules. A number of molecular techniques use either target amplification reactions (e.g., PCR, NASBA, TMA), or signal amplification reactions (e.g., bDNA, hybrid capture) (see Fred M. Ausubel, Roger Brent, Robert E. Kingston, David D. Moore, J. G. Seidman, John A. Smith, Kevin Struhl J., 2002. Current Protocols in Molecular Biology. John Wiley and Sons, New York.

As used herein, the term “nucleic acid or polynucleotide” refers to a linear sequence of covalently bonded nucleotides. The nucleotides are either a linear sequence of polyribonucleotides or polydeoxyribonucleotides, or a mixture of both. Examples of nucleic acid in the context of the present invention include—single and double stranded DNA, single and double stranded RNA, and hybrid molecules that have mixtures of single and double stranded DNA and RNA. Further, the nucleic acids of the present invention may have one or more modified nucleotides.

As used herein, the term “polymerase” refers to DNA polymerases, RNA polymerases and reverse transcriptases, which optimally perform nucleic acid chain elongation from 37 degrees Celsius to 80 degrees Celsius and more preferably from 55 degrees Celsius to 75 degrees Celsius.

Thermostable DNA polymerases include, but are not limited to, DNA polymerases from thermophilic Eubacteria or Archaebacteria, for example, Thermus aquaticus, T. thermophilus, T. bockianus, T. flavus, T. rubber, Thermococcus litoralis, Pyroccocusfuriousus, P. wosei, Pyrococcus spec. KGD, Thermatoga maritime, Thermoplasma acidophilus, and Sulfolobus spec. Preferable reverse transcriptases functional between 42-60 degrees Celsius includes, but are not limited to, MmLV reverse transcriptase, AMV reverse transcriptase, RSV reverse transcriptase, HIV-1 reverse transcriptase, and HIV-2 reverse transcriptase.

As used herein, the term “Polymerase Chain Reaction” or “PCR” means the application of cycles of denaturation, annealing with a primer and extension with a thermostable DNA polymerase, e.g. the Taq DNA polymerase, to amplify a target sequence of DNA. The PCR process for amplifying nucleic acid is described in the documents U.S. Pat. No. 4,683,195 and U.S. Pat. No. 4,683,202.

As used herein, the term “primer” refers to single-stranded oligonucleotides that are complementary to sequence portions on a template nucleic acid molecule separated by a variable number of nucleotides. Covalent bonding of nucleotide monomers can extend primers annealed to the template nucleic acid during amplification or polymerization of a nucleic acid molecule catalyzed by polymerases. Typically, primers are from 12 to 35 nucleotides in length and are preferably from 15 to 20 nucleotides in length. Primers are designed from known parts of the template, one complementary to each strand of the double strand of the template nucleic acid molecule, lying on opposite sides of the region to be synthesized. Primers can be designed and synthetically prepared as is well known in the art. Typically primers are used at concentrations of from 0.1 to 1 micromolar. Primers can be composed of either a DNA or an RNA molecule.

As used herein, the term “primer extension” refers to an in vitro method wherein a primer hybridized to an complementary sequence part of a single-stranded nucleic acid template molecule is extended by sequential covalent bonding of nucleotides to the 3′ end of the primer forming a new DNA molecule complementary to the DNA template molecule. The primer extension method transforms a single-stranded nucleic acid template into a partially or completely double-stranded nucleic acid molecule. The primer extension method as used herein is a single step nucleic synthesis process without amplification of the copy number of the template nucleic acid molecule.

As used herein, the term “probe” refers to an RNA or DNA molecule (or a derivative) attached to a detection mechanism (such as a fluorescent, enzymatic, immunoreactive, or radioactive reporter molecule) that allows visualization of the molecule when it has annealed with a complementary DNA or RNA molecule. As used in the present invention, a probe refers to an oligonucleotide which is designed to specifically hybridize to HPV nucleotides. Also, a probe used within the framework of the present invention will preferably be a detection probe, without excluding the other types of probes (e.g., detection) from this context.

As used herein, the term “simultaneous” or “concurrent” refers to existing or occurring at the same time within a defined space. In particular, the simultaneous determination of the presence of both HPV RNA and DNA refers to detection methods that will detect both the presence of target DNA and RNA molecules within a single reaction vessel. The term “simultaneous” also refers to detection methods that may occur at different time via different experimental protocols, however all experimental protocols and detection methods are performed within the same reaction vessel.

As used herein, the term “target sequence” of a probe or a primer is a sequence or part of a sequence within the HPV polynucleic acids to which the probe or the primer is completely complementary or partially complementary (i.e. with some degree of mismatch). It is to be understood that the complement of said target sequence is also a suitable target sequence in some cases. A probe should be complementary to at least the central part of its target sequence and in most cases is completely complementary to its target sequence. The term “type-specific target sequence” refers to a target sequence within the polynucleic acids of a given HPV type that contains at least one nucleotide difference as compared to any other HPV-type.

As used herein, the term “template” refers to a double-stranded or single-stranded nucleic acid molecule, which serves as a substrate for nucleic acid synthesis. In the case of a double-stranded DNA molecule, denaturation of its strands to form a first and a second strand is performed before these molecules may be used as substrates for nucleic acid synthesis. A primer, complementary to a portion of a single-stranded nucleic acid molecule serving as the template is hybridized under appropriate conditions and an appropriate polymerase may then synthesize a molecule complementary to the template.

This invention is further illustrated by the following examples that should not be construed as limiting. The contents of all references, patents and published patent applications cited throughout this application are incorporated herein by reference. Other terms used in the fields of molecular and cell biology and the DNA recombination as used herein should be generally well understood by the person of ordinary skill in the applicable arts.

### DETAILED DESCRIPTION OF THE INVENTION

Cervical carcinomas are the second most common cancer in women, following only breast cancer. Screening for malignant and premalignant disorders of the cervix has been historically performed according to the Papanicolaou cervical smear (PAP test). The cervical smears are examined by light microscopy and the specimens containing morphologically abnormal cells are classified on a scale of increasing severity of the lesion. Carcinoma of the cervix is unique in that it is the only solid tumor in which HPV DNA has been linked to the development of the lesion in virtually all cases worldwide. Because HPV infections are associated with the development of cervical cancer and associated neoplastic precursor lesions (Zur Hausen 2002), detecting HPV DNA in an appropriate sample has been suggested as a potential diagnostic means for the early detection of cervical cancer. Methods for the detection of HPV DNA, such as by the polymerase chain reaction (PCR) and hybrid capture (Digene Corporation, Gaithsberg Md.) are well know. Since a significant majority of women infected with HPV will clear the virus without any negative pathological consequences, there are still lingering questions as to the effectiveness of HPV DNA testing (Nobbenhius et al. 2001; Cuschieri et al. 2004).

The presence of HPV DNA is not necessarily an indication of an active infection. The presence of RNA transcripts (mRNA) has been suggested to be a much better indication of active and persistent infections, especially those transcripts from the E6 and E7 oncogenes (Ylitalo et al. 2000).

Methods for the detection of HPV RNA, such as the polymerase chain reaction using a reverse transcriptase enzyme (RT-PCR), and nucleic acid sequence based amplification (NASBA), are well know. Since HPV can exist in an inactive or latent state (i.e., with little or nor transcription), an assay designed to detect only HPV RNA would potentially miss an HPV viral infection which, at a later date, could become active. Thus, there exists a need for a diagnostic assay which determines the presence of both HPV DNA and HPV RNA in a patient sample.

There are multiple ways to distinguish between the presence of HPV RNA and DNA in a sample. Due to the physiological differences between DNA and RNA, there are a number of different experimental protocols which one could employ to differentiate between specific DNA and RNA molecules in a sample. Some examples of such differences are, but not limited to:
1. Single-stranded RNA versus double-stranded DNA
2. Stringencies of probe binding to RNA versus DNA (hybridization conditions)
3. RNA transcripts comprised of “sense” sequence by definition, but DNA is comprised of both “sense” and “anti-sense” sequence
4. Differential susceptibilities to destructive reagents (e.g. NaOH destroys RNA but not DNA; RNases destroy RNA but not DNA; DNases destroy DNA but not RNA)
5. Transcribed target regions (RNA and DNA) versus non-transcribed regions (DNA only)
   a. Non-transcribed examples: HPV long control region (LCR) or HPV promoter regions
   b. Transcribed examples: regions of HPV from L1, L2, E1, E2, E4, E5, E6 or E7 HPV genes
6. Specificity of polymerase enzymes for extension of primers (preferentially or exclusively) from RNA (RNA-dependent polymerases) or from DNA (DNA-dependent polymerases)
7. Intron (DNA-only) versus splice junctions (RNA-only); Contiguous versus non-contiguous target sequences; RNA splicing brings non-contiguous sequences together from exon junctions and these splice junctions are specific targets for only RNA; intron sequences are only present in DNA and are therefore specific targets for only DNA
   a. for example, E6 210-413 splice junction or E1-E4 877-3295 splice junction of HPV 31
8. Specific modifications; many RNA transcripts contain 3′ poly-A tail; DNA does not have a 3′ poly-A tail

In one embodiment of the invention, methods are used to detect nucleic acids associated with HPV, which in turn provides a basis for medical diagnosis and treatment. Methods of the invention solve the problems in the art through the detection of both HPV RNA and DNA at the same time from the same specimen sample rather than independently from two different samples from the same specimen.

In another embodiment of the invention, methods are provided for detecting the presence of specific HPV target nucleic acids in a biological sample using RNA and DNA probes. Preferred methods comprise suspending sample cells in a solution; isolating total nucleic acid from the sample cells (e.g., MagNA Pure LC Total Nucleic Acid Isolation Kit (Roche Applied Science)); contacting a target DNA sequence with at least one probe that is substantially complementary to at least a portion of said target nucleic acid, the detection of which is desired; contacting a target RNA sequence with at least one probe that is substantially complementary to at least a portion of a nucleic acid, the detection of which is desired; and detecting hybridization between the probes and the target sequences.

Cell samples may be obtained from the patient by a variety of techniques including, for example, by scraping or swabbing an area, or by using a needle to biopsy solid tumors or to aspirate body fluids from the chest cavity, bladder, spinal canal, or other appropriate area.

Cells samples can be from any source of biological material that can be obtained from a living organism directly or indirectly, including cells, tissue or fluid. Nonlimiting examples of the sample include blood, urine, semen, milk, sputum, mucus, plueral fluid, pelvic fluid, sinovial fluid, ascites fluid, body cavity washes, eye brushing, skin scrapings, a buccal swab, a vaginal swab, a pap smear, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, a microbial culture, a virus, and samples of in vitro cell culture constituents. In particular, the method of the present invention can be used with cells collected from the cervix, the breast (including ductal lavage), urinary tract malignancies (both biopsy tissue samples and urine cytology smears), colon, lung, bladder, skin, larynx, esophagus, bronchus, lymph nodes, and haematological malignancies. The method of the present invention may additionally be employed in assessment of pre-malignant abnormalities of cervical squamous epithelial cells (squamous intra-epithelial lesion, SL) or pre-malignant abnormalities in other tissues.

Preferably, the solution for suspending the sample cells should either be a cell preservative or a cell fixative. An exemplary preservative solution has a water-miscible alcohol in an amount sufficient to preserve the cellular integrity of the cells, an anti-clumping agent in an amount sufficient to prevent the cells from clumping in the solution, and a buffering agent maintaining the solution, with the cells, at a pH range between about 2 to about 7. A commercially available example is PreservCyt® solution from Cytyc Corporation (Boxborough, Mass.). The PreservCyt® solution is described in co-owned U.S. Pat. No. 5,256,571, incorporated herein by reference.

Probes can be detected by any means know in the art. By way of non-limiting example, probes may be detected by using immunohistological, immunocytological, hybridization using fluorescence and/or immunoenzymatic, techniques as well as hydrometry, polarimetry, spectrophotometry (e.g., mass and NMR) and chromatography (e.g., gas liquid, high performance liquid, and thin layer). Probes may be labeled with a detectable marker such as a radioisotope, a fluorescent compound, an enzyme, or an enzyme co-factor or a fluorescence/quencher pair as a molecular beacon.

Molecular beacon probes may be employed in the methods of the present invention for direct visualization of hybridization between a probe and a target template.

Molecular beacon probes are oligonucleotides that form stem-loop structures that can report the presence of specific nucleic acids in homogenous solutions (Tyagi S, Nature Biotechnology 1996; 14: 303-308.). They are useful in situations where it is either not possible or desirable to isolate the probe-target hybrids from an excess of the hybridization probes, such as in real time monitoring of polymerase chain reactions in sealed tubes or in detection of RNAs within living cells. The stem is formed by the annealing of complementary arm sequences on the ends of the probe sequence. The arm sequences are unrelated (i.e., not complementary) to the target sequence and each arm is labeled at its end. To one arm is attached a fluorescence moiety (i.e., at the 5′ terminal phosphate) and to the other a non-fluorescent quenching molecule (i.e., at the 3′ terminal hydroxyl group). In its nascent state, the molecular beacon emits no fluorescence. This is because the fluorescent-quencher pair is selected such that energy gained by the fluorophore is transferred to the quencher and is dissipated as heat, an occurrence that is referred to as fluorescence resonance energy transfer (FRET). At temperatures slightly above Tₘ, the stem portion of a molecular beacon unfolds and exposes the probe section of the molecule to target strands. Once exposed, the beacon and target hybridize.

Upon hybridization, a molecular beacon interacts with the target and undergoes a conformational change whereby the arm sequences of the beacon are forced apart such that the fluorophore and the quencher are physically distant from each other and their original positions. When the fluorophore is no longer in the proximity of the quenching molecule, FRET is no longer possible, and the fluorophore will then emit detectable light of appropriate wavelength when excited. When the probe is constructed as a molecular-beacon molecule, having a loop portion of nucleic acid complementary to target nucleic acids, the hybridization between the probe and the target template can be detected using a fluorescence reader, either in “real-time” or “end-point” fashion. Examples of such fluorescence reader include the i-Cycler commercially available from Bio-Rad Laboratories (Hercules, Calif.) and the Roche Light Cycler commercially available from Hoffmann-La Roche Ltd. (Basel, Switzerland).

In one embodiment of the invention, a molecular beacon probe comprising a stem-and-loop structure is constructed where the loop portion is a DNA sequence complementary to the target DNA, for example, a particular HIPV strain, to provide real time detection of the target DNA. The stem portions are not complementary to the target HPV sequence and provide FRET in the nascent state. The stem portions may be nucleotides other than DNA, such as RNA. Specific examples of fluorescent moieties are Texas Red®, Alexa Fluor, Oregon Green, Pacific Blue, fluorescein isothiocyanate (FITC), and Rhodamine Green.

In various embodiments, the nucleic acid probes can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

In yet another embodiment, the presence of the target DNA sequence in the sample cells is diagnostic of an HPV infection and the presence of the target RNA sequence in the same sample cells is diagnostic of an active HPV infection and may also be indicative of risks of cancer, such as risks associated with adenocarcinoma and squamous cell carcinoma of the cervix.

In still another embodiment, the presence of the target DNA sequence is indicative of the presence of a particular type of HPV. In more preferred embodiments, the target nucleic acid sequence is indicative of the presence of HPV strains selected from types 16, 18, 31, 33, 35, 39, 45, 51, 52, 56, 58, 59, 68 and 70.

In another embodiment, methods of the invention further comprise capturing the target nucleic acid onto a solid support using DNA-DNA, DNA-RNA, or RNA-DNA interactions.

Alternative embodiments of the invention include methods for detecting the presence of a target nucleic acids of HPV in a sample, comprising: capturing target DNA sequences onto a solid support with at least one probe that is substantially complementary to at least a portion of target DNA sequences of one or more HPV types, capturing target RNA sequences onto a solid support with at least one probe that is substantially complementary to at least a portion of target RNA sequences of one or more HPV types, and then detecting hybridization between the probes and the target nucleic acids. Other embodiments relate to methods for in situ detection of target DNA and RNA sequences of HPV in a sample by transferring suspended sample cells uniformly onto a surface; in situ hybridizing target sequences of a HPV contained in the cells with at least one probe that is substantially complementary to portions of a target DNA sequence and a target RNA sequence of one or more HPV types; and detecting hybridization between the probes and a target sequences.

Another embodiment of the invention provides methods of detecting target nucleic acids comprising amplifying target sequences. Such methods generally comprise providing a sample that may comprise DNA and RNA target sequences from at least one selected strain of an organism. Also provided are multiple primers substantially complementary to portions of target sequences from the selected strains of the organism. The sample is exposed to at least one probe that is sufficiently complementary to a portion of a DNA target sequence and to at least one analog probe that is sufficiently complementary to a portion of a RNA target sequence. The nucleic acid from at least one selected strain is amplified between the multiple primers and detection of such amplification product indicates the presence of at least one target nucleic acid in the sample.

Primers may be labeled with a label of choice (e.g. biotin). The amplification method used can be either polymerase chain reaction (PCR; Saiki et al., 1988), ligase chain reaction (LCR; Landgren et al., 1988; Wu & Wallace, 1989; Barany, 1991), nucleic acid sequence-based amplification (NASBA; Guatelli et al., 1990; Compton, 1991), strand displacement amplification (SDA; Duck, 1990; Walker et al., 1992), Transcription Mediated Amplification (TMA; CS Hill, Expert Rev Mol Diagn 1 (4):445-455, November 2001), or amplification by means of Q-beta replicase (Lizardi et al., 1988; Lomeli et al., 1989) or any other suitable method to amplify nucleic acid molecules known in the art.

The oligonucleotides used as primers or probes may also comprise nucleotide analogues such as phosphorothiates (Matsukura et al., 1987), alkylphosphorothiates (Miller et al., 1979) or peptide nucleic acids (Nielsen et al., 1991; Nielsen et al., 1993) or may contain intercalating agents (Asseline et al., 1984). As most other variations or modifications introduced into the original DNA sequences of the invention these variations will necessitate adaptations with respect to the conditions under which the oligonucleotide should be used to obtain the required specificity and sensitivity. However, the eventual results of hybridization will be essentially the same as those obtained with the unmodified oligonucleotides. The introduction of these modifications may be advantageous in order to positively influence characteristics such as hybridization kinetics, reversibility of the hybrid-formation, biological stability of the oligonucleotide molecules, etc.

In still another embodiment of the present invention, methods are used to selectively amplify high-risk strains of an infectious organism. In such methods, consensus primers for amplification of a preselected region of the infectious organism's DNA and RNA are used. Alone, these primers are capable of amplifying target sequences from most or all of the strains or variants of the disease organism. However, in methods of the invention, amplification of non-selected strains or variants is blocked such that any amplicon produced is representative of only the unblocked strains or variants. For example, methods of the invention may be used to selectively detect active infections of high-risk strains of HPV. An amplification reaction is conducted in the presence of HPV consensus primers and one or more nucleic acid blocking probes that hybridize only to a region at or between the primers in non-selected low-risk strains of HPV. The blocking probes prevent amplification of DNA and/or RNA target sequences from low-risk strains (i.e., full-length amplification between primers is prevented), while DNA and/or RNA target sequences from high-risk strains to which the blocking probes do not hybridize are amplified.

Several PNA probes have been shown to cross-hybridize with nucleic acids from multiple HPV strains. Thus, in some instances, a single probe or a mixture of probes may be used to screen for the presence of multiple strains in a single sample. Also according to the invention, a PNA probe which hybridizes only to nucleic acid of a single HPV strain may be used to specifically identify and/or type that strain in a sample. Such highly specific probes can be used in series or in parallel by, for example, utilizing differentiable markers attached to the probes. A mixture of such highly specific probes or a mixture of such highly specific probes and cross-hybridizing probes may be used to identify multiple strains of HPV, preferably all the high-risk ones and only the high-risk ones.

Hybridization screening as described above also may be performed in a preservative solution to combine cytological screening of a patient sample (e.g., a PAP smear) with HPV screening. An exemplary preservative solution has a water-miscible alcohol in an amount sufficient to fix mammalian cells, an anti-clumping agent in an amount sufficient to prevent the cells from clumping in the solution, and a buffering agent maintaining the solution, with the cells, at a pH range between about 2 to about 7. A commercially available example is PreservCyt® solution from Cytyc Corporation (Boxborough, Mass.). The PreservCyt® solution is described in co-owned U.S. Pat. No. 5,256,571, incorporated herein by reference.

According to methods of the invention, PNA probes are designed to be substantially complementary to portions of nucleic acids of one or more HPV types. HPV DNA sequences can be found in numerous public databases, such as GENBANK. PNA probes for use in the invention can be designed to hybridize to homologous regions of one or more HPV strains, for example, homologies that are common among high-risk HPV strains but not common among low-risk HPV strains are selected to ensure the specificity of PNA probes to high-risk types of HPV DNA only. A preferred region of homology is the HPV L1 consensus region. The L1 region has been known to encode for the major capsid protein of HPV and is retained whenever viral integration occurs in a cell. Other regions in the viral genome, e.g., the E6 open reading frame as known in the art, described in U.S. Pat. No. 5,888,724 to Silverstein et al. and incorporated herein by reference, can also be used to generate homologies.

DNA sequences of the L1 regions of various HPV types can be downloaded from GENBANK. L1 regions of high-risk HPV types and then compared using Vector NTI software (Informax Inc., North Bethesda, Md.) to establish areas of homology. Thereafter, areas of homology that are common among known high-risk types of HPV but not common among low-risk types of HPV can be selected. This is to ensure that any PNA probe so constructed would exhibit maximal specificity for the high-risk types of HPV but minimal or no specificity for the low-risk types of HPV. The selected sequences can then be then analyzed with the Vector NTI program to discard any sequence that would form significant secondary structure (e.g., hairpins).

Typically, the probes should be short enough to stay in solution, i.e., less than 18 or 19 bases. In constructing the probes, preferably for any stretch of 10 bases in a sequence, there are no more than 6 purines. Also, 4 to 5 purines in a row, especially 3 guanines in a row, preferably should be avoided. A preferred length of a PNA probe is 8 or more units. The present invention also contemplates the use of hybrids between PNA and other nucleic acids, such as DNA and RNA, as probes for detecting multiple strains of interest. PNA-DNA chimeras, which is known in the art and described in Uhlmann, E., Biol. Chem. 1998, 379 (8-9): 1045-52, the disclosure of which is incorporated herein by reference, can be useful in methods of the present invention.

Compared to DNA or RNA, PNA has a higher affinity for a substantially complementary nucleic acid template. Therefore, in a situation where a PNA probe competes with a DNA or RNA primer for part or all of a hybridization site, hybridization between the PNA probe and the nucleic acid template is favored. As a result, full-length amplification between the hybridization sites of the primer set will be inhibited. See U.S. Pat. No. 5,891,625 to Buchardt et al., the entirety disclosure of which is incorporated herein by reference.

Once the sequence of a PNA probe is finalized, a probe can be synthesized, purified and labeled according to methods known to those skilled in the art. For example, such methods are disclosed in PCT publication WO92/20702, U.S. Pat. No. 5,539,082 to Neilsen et al. and U.S. Pat. No. 5,731,416 to Garner, all incorporated herein by reference to the extent applicable. Such synthesis, labeling (e.g., with biotin) and purification of PNA probes can also be obtained from commercial vendors such as PerSeptive Biosystems Inc. (Framingham, Mass.).

The present invention also includes kits that contain probes made in accordance with the invention or preservation solution or both, and may include other reagents needed for a detection assay. For example, a kit may include materials, instruments and devices for taking cervical samples, for storing the samples and for isolating and purifying genetic materials from the samples. An in vitro hybridization kit may include multiple probes, e.g., DNA probes and RNA made in accordance with the invention, and other reagents needed for hybridization reactions. Any of these probes may be labeled, e.g., with biotin or a fluorophore. Kits made in accordance with the present invention may further include enzymes, primers, buffers and other reagents for a nucleotide-amplification reaction. An in situ hybridization kit may include enzymes, fixation solution, buffers and other reagents needed for slide preparation, signal amplification and other techniques employed in situ hybridization. A kit may also include enzymes, antibodies, substrates and other reagents needed for labeling, detection and visualization of a target molecule. Such kits may also include probes and reagents needed for further HPV typing.

### EXAMPLES

Simultaneous Detection of HPV RNA and DNA

1. Structure Based Approach
a. Isolate total nucleic acid under non-denaturing conditions from a specimen
b. Hybridize a molecular beacon probe “A” (labeled with fluorophore “a”) to single-stranded RNA regions of transcribed HPV genes (e.g. L1, L2, E1, E2, E6 or E7)
c. Measure signal generated from fluorophore “a” with appropriate band-pass filters; this signal is proportional to the amount of RNA present
d. Analogous DNA sequence will not yield a signal since it is in a double-stranded form and therefore not accessible to probe “A”
e. Denature the nucleic acids in the reaction mixture
   i. Heat the solution to denaturation temperatures, or
   ii. Add denaturants, for example NaOH. This method of denaturation would require subsequent neutralization of the pH but has the benefit of destroying RNA present in the sample
f. Hybridize a molecular beacon probe “B” (labeled with fluorophore “b”) to anti-sense DNA strand of HPV genes (e.g. L1, L2, E1, E2, E6 or E7)
g. Measure signal generated from fluorophore “b” with appropriate band-pass filters; this signal is proportional to the amount of DNA present
h. Fluorescence produced from fluorophore “b” will not produce any signal from RNA because probe “B” is designed to hybridize to sequence of the opposite sense from RNA sequence.

2. Transcribed and Non-Transcribed Regions
a. Isolate total nucleic acid using denaturing conditions from a specimen
b. Hybridize a molecular beacon probe “A” (labeled with fluorophore “a”) to a RNA splice junction derived from a transcribed region of HPV (e.g. E6 210-413 splice junction or E1-E4 877-3295 splice junction of HPV 31)
c. Simultaneously, hybridize a molecular beacon probe “B” (labeled with fluorophore “b”) to a non-transcribed region of HPV DNA (e.g. the long control region at terminus of HPV genome).
d. Measure signal generated from fluorophore “a” with appropriate band-pass filters; this signal is proportional to the amount of RNA present.
e. Measure signal generated from fluorophore “b” with appropriate band-pass filters; this signal is proportional to the amount of DNA present

3. Location Specific Capture
a. Bind a capture probe specific for DNA to a portion of the surface of a reaction vessel (e.g. the bottom of a microtiter well; glass surface). The probe may ideally be complementary to a non-transcribed region of HPV (e.g. the long control region at the terminus of HPV genome).
b. Bind a capture probe specific for RNA to a different portion of the surface of the same reaction vessel. The probe may hybridize specifically to a RNA splice junction or to the poly-A tail.
c. Add denatured nucleic acid from a test specimen
d. Under hybridization conditions, bind test nucleic acid to capture probes on solid phase
e. Add and hybridize a fluorescently labeled probe to the surface-bound test nucleic acid. The labeled probe may bind to specific sequences of the RNA or DNA target or may bind equally to both the RNA and DNA targets. (Alternatively, the probe may be labeled with an enzyme activity—such as HRP or AP—and visualized through the use of a precipitating chromogenic substrate).
f. Visualize resulting fluorescence signals by a means that has the appropriate resolution to spatially distinguish the two portions of the surface of the reaction vessel.
g. Discrimination between RNA and DNA is dependent on the position of the signal on the solid surface.

4. Amplified Assay Format
a. Isolate total nucleic acid (optional—amplification directly from a specimen lysate is feasible)
b. Perform RT-PCR reaction to amplify both HPV RNA and DNA. A single set of primers may be used for this reaction and should be designed within exons such that they span an intron. For example, for the HPV 31 and primer set could be designed within the E6 gene and span the 210-413 splice junction. The 3′ end of upstream primer should bind at least 10 nucleotides upstream of the 210 junction (e.g. 5′-185 to 205-3′) and the 3′ end of the downstream primer should bind at least 10 nucleotides downstream of the 413 splice junction (e.g. 3′-428 to 448-5′).
c. Following amplification, the amplicons derived from HPV DNA will include the intron sequence. The amplicons derived from HPV RNA will not include the intron sequence but will contain the splice site junction.
d. The amplicons derived from the RNA and DNA targets may be distinguished based on amplicon length (those from DNA will be longer than those from RNA).
e. Alternatively, the amplicons derived from RNA and DNA may be distinguished through the use of molecular beacons—one which binds specifically to intron sequence and therefore will detect amplicons from DNA targets and one which binds specifically to splice junction sequence and therefore will detect amplicons from RNA. In this case, the molecular beacons should be differentially labeled with two distinct fluorophores which can be optically and separately detected.

In the above examples, specific nucleotide sequences of potential HPV probes, capture probes and/or primers have not been specified, as one normally skilled in the art can appropriately design such materials from the published HPV genomic sequences and HPV transcription maps.

RNA splice site probes are designed such that the splice junction is more or less centered in the middle of the probe. Furthermore, the probe length is designed such that the Tm of the probe to either “half of the splice site junction” under the chosen hybridization conditions will not result in a binding reaction. In other words, only when the two halves are brought together by a splicing reaction, will the splice site junction probe bind.

Molecular beacon probes do not necessarily have to be added as a separate step. Such probes can be incorporated into a PCR mixture and present during amplification to permit real time detection of the reaction.

According to another aspect of the invention, methods and materials are provided for clinical assay of a biological sample to identify one or more selected nucleic acids within the same reaction vessel. In a preferred embodiment, methods are used to detect one or more selected strains or variants of a disease organism, such as the DNA and RNA of a bacterium, yeast, other microbe, or a virus. In a preferred embodiment of the invention, methods are provided for detecting the presence of specific HPV target nucleic acids in a biological sample using RNA and DNA probes. Preferred methods comprise suspending sample cells in a solution; isolating total nucleic acid from the sample cells; contacting a target DNA sequence with at least one probe that is substantially complementary to at least a portion of said target nucleic acid, the detection of which is desired; contacting a target RNA sequence with at least one probe that is substantially complementary to at least a portion of a nucleic acid, the detection of which is desired; and detecting hybridization between the probes and the target sequences. The presence of probes indicates presence of an active HPV infection.

In a preferred embodiment, a gynecological cell sample is obtained from a female subject to screen for strains of HPV that indicate high-risk of cervical cancer. Such a sample may be suspended, for example, in a PreservCyt® preservative solution. Total nucleic acid content (under non-denaturing conditions) from the sample is isolated and purified using kits and methods well known to one skilled in the art.

In one method embodying the invention, a pair of molecular beacons, for example, a FITC-labeled RNA (Probe A) sequence, and a Texas Red® labeled DNA sequence (Probe B), are selected as probes for a labeling reaction. Probe B binds to portions of the HPV L1 consensus region. The L1 consensus region is retained whenever viral integration occurs in a cell. Probe A binds to a region in a HPV mRNA, such as the L2, E1, E6 and E7 transcripts.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for the simultaneous detection of human papiloma virus (HPV) DNA and RNA sequences in a sample, the method comprising;
a) obtaining a sample of biological material containing a target nucleotide sequence and isolating total nucleic acid from said sample
b) detecting the presence of HPV DNA and RNA simultaneously in said sample
wherein the presence of HPV DNA is indicative of HPV in said sample and the presence of HPV RNA is indicative of an active HPV infection in said sample.

2. The method of claim 1, wherein sample is comprised of cervical cells.

3. The method of claim 1, wherein the presence of HPV DNA and RNA is detected by amplifying a target nucleic acid sequence in said sample.

4. The method of claim 1, wherein the presence of HPV DNA and RNA is detected by the binding of a probe to a target nucleic acid sequence in said sample.
